(19) United States
(12) Patent Application Publication
(10) Pub. No.: US 2011/0038943 A1
(43) Pub. Date: Feb. 17, 2011

(54) MILKWEED SEED OIL ADMINISTERED TO ANIMALS

(75) Inventor: Herbert D. KNUDSEN, Ogallala, NE (US)

Correspondence Address: CURATOLO SIDOTI CO., LPA, 24500 CENTER RIDGE ROAD, SUITE 280, CLEVELAND, OH, 44145, US

(73) Assignee: Natural Fibers Corporation, Ogallala, NE (US)

(21) Appl. No.: 12/854,513

(22) Filed: Aug. 11, 2010

Related U.S. Application Data
(60) Provisional application No. 61/233,574, filed on Aug. 13, 2009.

(51) Int. Cl. A61K 31/20; A61P 7/00; A61K 35/60

(52) U.S. Cl. 424/523; 514/558

(57) ABSTRACT

Milkweed seed oil administered to an animal improves the health of the animal. This improvement is evidenced by the positive change in the analytical results of blood tests for cholesterol after the animal has been administered an effective amount of milkweed seed oil as compared to the blood test results of the animal before the milkweed seed oil was administered.

### CROSS REFERENCE TO RELATED APPLICATIONS

This application claims the benefit of the filing date under 35 U.S.C. 119(e) from U.S. Provisional Application For Patent Ser. No. 61/233,574, filed on Aug. 13, 2009, which is hereby incorporated by reference in its entirety.

### TECHNICAL FIELD

A composition and method are provided for improving mammalian animal health. Blood tests for cholesterol are an important diagnostic tool in measuring animal health. Provided is a treatment option to improve animal health and blood test results by treating an animal with milkweed seed oil in addition to or in substitution of other conventional treatment options.

### BACKGROUND

Animal health is monitored by testing blood samples secured from the animal. Typically these tests involve the analysis of the total cholesterol, the HDL (good cholesterol), the triglycerides, the LDL (bad cholesterol), the ratio of the total cholesterol divided by the HDL, the glucose concentration and the ratio of HDL divided by the LDL. For human beings, this information is analyzed by medical professionals to determine what diet, exercise and medication should be recommended to the patient.

A wide range of potential solutions to unacceptable numbers in an animal's blood test results are now available.

### DETAILED DESCRIPTION

An additional avenue of treatment is provided to produce improved and/or acceptable animal blood test results.

Milkweed seed oil is administered to mammalian animals to improve their health. The improvement in health is determined by the results from blood test results measuring cholesterol. The milkweed seed oil formulation may be administered to mammalian animals for non-therapeutic or therapeutic applications.

Milkweed seed oil as disclosed herein is a product comprised primarily of fatty acids and including small quantities of other ingredients. The milkweed seed oil is administered topically, orally or internally to an animal in an amount and for a period of time that positively affects the animal's blood tests for cholesterol.

Milkweed seed oil according to an embodiment is produced from the seeds found in the pods of milkweed plants, which are known under the Latin binomial name Asclepias. Any of the numerous Asclepias plants can be used as a source of seed for producing milkweed seed oil. The most prominent sources of milkweed seeds in the work on certain embodiments to date are seeds from Asclepias Syriaca and Asclepias Specosia. Other non-limiting sources of milkweed seed oil may include, for example, Asclepsia albicans, Asclepias amplexicaulis, Asclepias californica, Asclepias cordifolia, Asclepias cryptocerus, Asclepias curassayica, Asclepias eriocarpa, Asclepias erosa, Ascelpias exaltat, Asclpeias fascicularis, Ascelpias fascicularis, Asclepias fruticosa, Asclepias humistrata, Asclepias incarnata, Ascelpias lanceolata, Ascelpias linaria, Asclepias linearis, Asclepias meadii, Asclepias nyctaginifolia, Ascelpias obovata, Ascelpias physiocarpa, Asclepias purpurascens, Asclepias quadrifolia, Asclepias solanoana, Asclepias speciosa, Asclepias subulata, Asclepias sullivantii, Asclepias syriaca, Asclepias tuberose, Asclepias variegate, Asclepias verticallata, Asclepias vestita, Asclepias and vincetoxicum. This list of Asclepias species is not exhaustive as scientists believe that there may be over 800 different varieties of Asclepias.

Production of Milkweed Seed Oil. In certain embodiments, milkweed seed oil is produced by cold pressing substantially clean milkweed seeds in a seed press or by extracting the oil from the seed with a non-polar solvent to recover the milkweed seed oil. Suitable non-polar solvents include but are not limited to hexane, carbon dioxide and petroleum ether. Extraction with these solvents produces a liquid lipid product comprising primarily fatty acids, and in certain embodiments, a liquid lipid product consisting of fatty acids.

Milkweed seed may also contain toxic cardenolides that can be recovered from the milkweed seed with a polar solvent as a powder or as a dissolved solid in the polar solvent. Non-polar solvents used in extracting the fatty acids from the seed may be selected so that the effectiveness of the milkweed seed oil is not impaired by the presence of cardenolides. Rogers E. Harry-Okuru and Thomas P. Abbott in Industrial Crops and Products 7 (1997) 53-58 concluded that “Cold-pressed and solvent (petroleum ether) extracted milkweed oils show no detectable cardenolides by the alkaline TNBP method at a limit of less than 1.0 ppm.” In one embodiment, milkweed seed oil is produced that has no detectable cardenolides at a limit of less than 1 part per million.

Raw milkweed seed oil produced in pressing equipment or in extraction with a solvent may need to be filtered to remove fine particles of the milkweed seed. This can be accomplished with traditional seed oil separating equipment, efficient filtration systems or with other known means of separation.

A typical fatty acid profile of milkweed seed oil produced in a seed press is shown below in Table 1. As can be deduced from the data, the milkweed seed oil had a large majority of fatty acids commonly found in food grade vegetable oils. The oil also contained two less common fatty acids, C16:1 palmitoleic acid and C18:1 cis-vaccenic acid. The milkweed seed oil with the fatty acid profile in Table 1 was used in the experiments described below.

TABLE 1
Milkweed Seed Oil Fatty Acid Profile
>1% Concentration

| | |
|---|---|
| 16:0 Palmitic Acid | 6.6% |
| 16:1 Palmitoleic Acid | 11.8% |
| 17:0 Margaric | 2.4% |
| 18:1 n-7 cis-Vaccenic Acid | 12.4% |
| 18:1 n-9 cis-Oleic Acid | 23.9% |
| 18:2 Linoleic Acid | 40.4% |
| 20:0 Arachidic Acid | 1.3% |

In addition to the fatty acids shown in the table above, a minor amount of other fatty acids which are present at concentrations below 1% of the oil were in the milkweed seed oil recovered. Milkweed seed oil also contains other ingredients in trace amounts. Vitamins, phytosterols, carotenes and other trace ingredients found in milkweed seed oil may have a positive impact on animal health even at very low concentrations.

Milkweed seed used to produce milkweed seed oil differs based on the genetic line of Asclepias used in production, the weather during the milkweed pod growth and the geographical region where the milkweed was grown. These variations, however, produce seed with oil contents within the limits of the current composition and method.

Also, milkweed seed oil may be produced not only from milkweed seed, but also from milkweed seed oil press cake. Press cake is the solid discharge from a seed press after some but not all of the oil is pressed out of the seed. The milkweed seed oil press cake can be rerun in a press to produce more milkweed seed oil or the press cake can be solvent extracted. Press cake extractions may provide unique, but similar fatty acid profiles to the data shown in Table 1. Milkweed seed oil from these sources are effective according to the present composition and method, provided that liquid fatty acid oil is recovered and that cardenolides are not present in the oil at a level that is harmful to the animal, in most embodiments such cardenolides being below 1 part per million.

Factors Affecting the Application of Milkweed Seed Oil to Animals. A method is provided for improving the health of a mammalian animal. The method comprises administering an effective amount of the disclosed milkweed seed oil formulation. Milkweed seed oil may be administered to animals topically, orally or internally. In certain embodiments, the amount administered daily measured as 100% milkweed seed oil is less than about 5 grams per kilogram of body weight, and in some embodiments is less than one gram per kilogram of body weight. Lower amounts of milkweed seed oil, such as about 0.1 gram per kilogram of body weight and even about 0.01 gram per kilogram of body weight have been proven effective.

For topical applications, milkweed seed oil can be administered to any area of the skin. The milkweed seed oil penetrates through the skin quickly. One method is to administer milkweed seed oil onto the feet of the animal. The milkweed seed formulation may be incorporated into a wide variety product forms for topical administration to a subject. By way of illustration, but not it limitation, the milkweed seed oil may be incorporated into topical creams, gels, lotions, ointments, sprays (aerosol and non-aerosol sprays), and dermal patches for topical administration of the milkweed seed oil formulation.

The milkweed seed oil liquid formulation administered to an animal may be milkweed seed oil alone or milkweed seed oil in combination with other ingredients that do not block the beneficial effect of milkweed seed oil. Formulated compositions of liquid milkweed seed oil and other components are included as embodiments of the present composition and method. The concentration of milkweed seed oil in the formulations of certain embodiments comprises about 4 weight % or more of milkweed seed oil, with formulations containing about 10 weight % or more of milkweed seed oil being other useful embodiments, and formulations containing about 15 weight % or more of milkweed seed oil being especially useful embodiments.

Optional compatible ingredients in milkweed seed oil liquid formulations include at least one of food grade vegetable oils, nut oils, fish oils, or fragrances. Suitable vegetable oils include but are not limited to at least one of olive oil, canola oil, safflower oil, corn oil, soybean oil, sunflower seed oil, or flax oil. Suitable nut oils include but are not limited to at least one of walnut oil, almond oil, or macadamia nut oil. A wide range of fragrances are available commercially that may be used to alter the odor of the milkweed seed oil product formulation. According to certain illustrative embodiments, the milkweed seed oil formulation comprises milkweed seed oil and fish oil. The milkweed seed oil formulation containing milkweed seed oil and fish oil may comprise greater than 0 to about 10 mg of milkweed seed oil and the fish oil. By example, the milkweed seed oil formulation may include a combination greater than 0 about 10 mg of milkweed seed oil and about 700 mg of fish oil.

The dosage of the milkweed seed oil administered to an animal depends on the concentration of the milkweed seed oil in the formulation. In topical applications using 100% milkweed seed oil, excellent results have been obtained on a 93 kilogram animal using a dose of 0.3 gram per application 2 times per day in separated periods of time. Administering this amount to the animal is a daily amount of 0.006 grams per kilogram of body weight. This dosage is within a preferred daily rate of administration—less than 0.01 grams per kilogram of body weight. The same amount of milkweed seed oil could be administered using one gram doses of a blend containing 30% milkweed seed oil and 70% vegetable oil.

Evidence indicates that total daily use of less than 20 grams of milkweed seed oil, calculated as 100% milkweed seed oil, is more than sufficient to produce the desired improvement in the blood tests in a 100 kilogram animal. The dosage of milkweed seed oil taken orally or internally rather than topically may vary from these general guidelines for topical applications. Experience has shown that the animal administered milkweed seed oil topically demonstrates the impact of the formulation on the body of the animal. Changes in the dosage may be necessary in response to these observations.

The optimum milkweed seed oil applications have not been fully evaluated. The best data points to date are the experimental tests below. Dramatic and unexpected improvements of the blood test results for cholesterol were achieved in humans. Higher dosages or longer usage of milkweed seed oil could further improve the blood test results. It may be necessary to continue milkweed seed oil treatment to maintain the improved blood test results, but there is evidence that milkweed seed oil has a positive impact on blood tests for cholesterol for a few days after daily application or consumption terminates. The need to have an effective amount of milkweed seed oil may require the user to alter the dosage over time to provide the desired benefits as measured by the blood test results.

Oral administration of milkweed seed oil has also been found to be beneficial in lowering blood triglyceride levels in mammalian animals.

Milkweed seed oil may be formulated into a wide variety of orally ingestible compositions. Liquid forms include solutions, suspensions, emulsions, gels, syrups, liquid-containing capsules, and the like. According to certain embodiments, the liquid milkweed seed oil formulation is contained within an orally ingestible capsule. Upon ingestion, the liquid-containing capsule is digested and the liquid milkweed seed oil formulation is released from the capsule.

According to certain illustrative embodiments, the milkweed seed oil may be formulated with an orally ingestible liquid carrier to provide an orally ingestible milkweed seed oil. For example, the milkweed seed oil may be formulated with an orally ingestible liquid carrier to provide a beverage, dietary supplement formulation, or nutritional supplemental. The beverages, dietary supplements and nutritional supplements may be provided ready for oral ingestion or may be provided in a concentrate for that requires dilution with acceptable liquids prior to oral ingestion. According to other embodiments, the milkweed seed may be formulated into other solid orally ingestible product forms, such as powders, pills, lozenges, tablets, caplets, capsules, gel capsules, edible films, and the like. Flavoring agents may also be added to the orally ingestible products to provide a more palatable orally ingestible composition.

The orally ingestible milkweed seed oil formulation may further include nutritionally effective amounts of an additional agent. According to certain embodiments, the milkweed seed oil formulation may further comprise effective amounts of at least one vitamin, or at least one mineral or a combination of at least one vitamin and at least one mineral. According to certain embodiments, the milkweed seed oil formulation comprises a nutritionally effective amount of milkweed seed oil and a nutritionally effective amount of at least one vitamin. According to certain embodiments, the milkweed seed oil formulation may comprise a nutritionally effective amount of milkweed seed oil and a nutritionally effective amount of at least one vitamin. According to certain embodiments, the milkweed seed oil formulation may comprise a nutritionally effective amount of milkweed seed oil and a nutritionally effective amount of at least one vitamin. According to certain embodiments, the milkweed seed oil may comprise a nutritionally effective amount of milkweed seed oil formulation and a nutritionally effective amount of at least one vitamin and at least one mineral. The milkweed seed oil formulation may also include at least one amino acid alone or at least one amino acid in combination with at least one vitamin and/or at least one mineral.

Experimental Production of Milkweed Seed Oil in a Seed Press. In this experiment, clean milkweed seed from Asclepias Syriaca and Asclepias Specosia plants was pressed in a screw press to produce raw milkweed seed oil. The raw oil was strained in two layers of high thread count cotton fabric at atmospheric pressure to produce clean milkweed seed oil. The clean milkweed seed oil was placed in a marked container, sealed and placed in a constant temperature room maintained at about 70 degrees farenheit. Samples of milkweed seed oil for the experiments described below were taken from the stored oil and maintained in marked sealed bottles. The fatty acid profile of milkweed seed oil produced in this experiment is shown above in Table 1.

Experimental Testing of 100% Milkweed Seed Oil Administered to a 205 Pound Male. A test to determine the impact of milkweed seed oil treatment was conducted on a 67 year old male human being weighing 205 pounds (93 kg). For two years before the test began, the male had been taking 40 mg of Lipitor® medication, 6.25 mg of Hyzaar® medication, one aspirin tablet and vitamin supplements daily. During that two year period previous to the test, the male ate a regular healthy diet without restriction and exercised for 15 to 25 minutes per day. No attempt was made to lose or gain weight. During the two year period before the test, the male's normal weight ranged from 204 to 209 pounds (93-95 kg). During the two year period before the test in this experiment, the blood analysis from annual physicals on the male was generally stabilized at the “Before” column levels shown in Table 2.

At the beginning of the test of the milkweed seed oil by topical application, the weight of the male was taken and recorded at 205 pounds (93 kg). Throughout the test period, the male maintained substantially the same diet, exercise routine and medication and supplement program described above for the two years before the test. During the test period, the male topically administered two doses of 100% milkweed seed oil to his body every day. One dose was administered in the morning and one at night before going to bed. For the topical application he placed 6 drops, 0.3 gram, of milkweed seed oil into one hand, rubbed both hands together to disburse the oil on the two hands and administered the milkweed seed oil to the bottom of both of his feet and in between his toes. A daily amount of 0.006 grams per kilogram of original body weight was administered in this experiment. This dosage is within the most preferred daily rate of administration—less than 0.01 grams per kilogram of body weight. The test applying milkweed seed oil to the 205 pound man as described was continued for 66 days. At the end of the 66 day test administering milkweed seed oil, the male weighed 199 pounds (90 kg), six pounds (2.7 kg) less than at the beginning of the test. The lipid data before and after the test are shown in Table 2.

TABLE 2
205 Pound Male Experiment Comparison of
Blood Test Results After vs. Before Test

| | After | Before | After/Before |
|---|---|---|---|
| Total Cholesterol | 104 | 146 | 71% |
| HDL | 61 | 62 | 98% |
| Triglycerides | 80 | 129 | 62% |
| LDL | 26 | 58 | 45% |
| TC/HDL | 1.7 | 2.4 | 71% |
| Glucose | 90 | 97 | 93% |
| HDL/LDL | 2.3 | 1.1 | 219% |

Discussion of Results. The dramatic improvement in the blood test results was unexpected. Total cholesterol was reduced by 29%, HDL, the good cholesterol, was essentially stable, triglycerides were down 38%, LDL, the bad cholesterol, was down 55%, total cholesterol divided by HDL was down 29%, glucose was down 7% and the HDL/LDL ratio more than doubled.

The male's blood test results for cholesterol show stability of HDL and improvement in each of the other test results shown in Table 2 according to modern medical standards. In light of these results a medical doctor reduced the male's prescription for Lipitor® from 40 mg per day to 20 mg per day.

Experimental Testing of 16% Milkweed Seed Oil Administered to a 202 Pound Female. A test to determine the impact of milkweed seed oil treatment was conducted on a 65 year old female human being weighing 202 pounds (92 kg). The female had been taking 5 mg of Crestor® medication and other medicines that do not impact the blood tests for cholesterol. Before the test, the female ate a regular healthy diet without restriction and was physically active but had no specified exercise plan. No significant attempt was made to lose or gain weight. Before the test in this experiment, the blood analysis on the female was generally stabilized at the “Before” column levels shown in Table 3.

Throughout the test period, the female maintained substantially the same diet, exercise routine and medication. During the test period, the female topically administered two doses of a formulation of milkweed seed oil and canola oil to her body, one in the morning and one at night before going to bed. The liquid milkweed seed oil formulation was comprised of 16% milkweed seed oil and 84% canola oil. For the topical application she placed one dropper full, 0.6 gram, of the milkweed seed oil formulation into one hand, rubbed both hands together to disburse the formulated milkweed seed oil on two hands and administered the milkweed seed oil to the bottom of both of her feet. Based on 100% milkweed seed oil, the daily consumption of milkweed seed oil was 0.19 grams per day. Based on her initial weight of 92 kg, the dosage was 0.002 grams per kilogram of body weight. This was less than half the dosage given to the male in the first experiment. The test applying milkweed seed oil to the female as described was continued for 14 days and was discontinued 5 days before the “After” test. Data for the test are shown in Table 3.

TABLE 3
202 Pound Female Experiment Comparison of
Blood Test Results After vs. Before Test

| | After | Before | After/Before |
|---|---|---|---|
| Total Cholesterol | 123 | 131 | 94% |
| HDL | 51 | 54 | 94% |
| Triglycerides | 161 | 122 | 132% |
| LDL | 41 | 53 | 77% |
| TC/HDL | 2.4 | 2.4 | 99% |
| Glucose | 131 | 108 | 121% |
| HDL/LDL | 1.2 | 1.0 | 122% |

Discussion of Results. The improvement in the blood test results especially the 23% reduction in LDL and the 22% improvement in the ratio of HDL to LDL were unexpected.

There are many possible treatments of animals with milkweed seed oil to improve the blood test results for cholesterol according to the compositions and methods discussed and exemplified herein. Although the embodiments have been described in detail through the above description and the preceding examples, these examples are for the purpose of illustration only and it is understood that variations and modifications can be made by one skilled in the art to identify and administer these treatments using milkweed seed oil without departing from the spirit and the scope of the disclosure. It should be understood that the embodiments described above are not only in the alternative, but can be combined.

What is claimed is:

1. A formulated composition for administration to an animal comprising milkweed seed oil and optionally other ingredients compatible with milkweed seed oil.

2. The formulated composition of claim 1 wherein the other ingredients comprise at least one of vegetable oils, nut oils, fish oils or fragrances.

3. The formulated composition of claim 2 wherein the formulated composition comprises milkweed seed oil and fish oil.

4. The formulated composition of claim 2 wherein the formulated composition comprises about 10 mg of milkweed seed oil and about 700 mg of fish oil.

5. The formulated composition of claim 1 wherein the concentration of milkweed seed oil is about 4% or more.

6. The formulated composition of claim 1 wherein the concentration of milkweed seed oil is about 10% or more.

7. The formulated composition of claim 1 wherein the concentration of milkweed seed oil is about 15% or more.

8. The formulated composition of claim 1 wherein the milkweed seed oil is produced by pressing the milkweed seed in a seed press.

9. The formulated composition of claim 1 wherein the milkweed seed oil is produced by solvent extraction of milkweed seed or milkweed seed oil press cake and the concentration of cardenolides in the milkweed seed oil is less than 1 part per million.

10. The formulated composition of claim 1, wherein said formulated composition comprises a liquid.

11. The formulated composition of claim 1, wherein said formulated composition comprises a solid.

12. The formulated composition of claim 1, wherein said ingredients compatible with milkweed seed oil comprise at least one of an orally acceptable carrier, a vitamin, a mineral, and an amino acid.

13. A method for improving the cholesterol blood test results of an animal by administering an effective amount of milkweed seed oil to the animal.

14. The method of claim 13 wherein the animal is a human being.

15. The method of claim 13 wherein the amount of milkweed seed oil administered to the animal per day is less than about 5 grams per kilogram of body weight.

16. The method of claim 15 wherein the amount of milkweed seed oil administered to the animal per day is less than about 0.1 grams per kilogram of body weight.

17. The method of claim 16 wherein the amount of milkweed seed oil administered to the animal per day is less than about 0.01 grams per kilogram of body weight.

18. The method of claim 13, wherein said administering comprises orally administering said therapeutically effective amount of the milkweed seed oil formulation.

19. The method of claim 13, wherein said administering comprises topically administering said therapeutically effective amount of the milkweed seed oil formulation.
